(19) United States
(12) Patent Application Publication
(10) Pub. No.: US 2010/0221327 A1
(43) Pub. Date: Sep. 2, 2010

(54) Nanoparticulate azelnidipine formulations

(75) Inventors: Scott Jenkins, Downingtown, PA (US); Gary G. Liversidge, West Chester, PA (US)

Correspondence Address: Elan Drug Delivery, Inc. c/o Foley & Lardner, 3000 K Street, N.W., Suite 500, Washington, DC, 20007-5109, US

(73) Assignee: Elan Pharma International Limited

(21) Appl. No.: 11/452,421

(22) Filed: Jun. 14, 2006

Related U.S. Application Data
(60) Provisional application No. 60/690,716, filed on Jun. 15, 2005.

(51) Int. Cl. A61K 9/52; A61K 9/14; A61K 9/48; A61K 9/20; A61K 9/22; A61K 31/397; C07D 401/12; A61P 9/12; A61P 9/10

(52) U.S. Cl. 424/457; 424/400; 424/451; 424/464; 424/468; 514/210.2; 546/268.1; 977/773

(57) ABSTRACT

The present invention is directed to compositions comprising a nanoparticulate azelnidipine, or a salt or derivative thereof, having improved bioavailability. The nanoparticulate azelnidipine particles of the composition have an effective average particle size of less than about 2000 nm and are useful in the treatment of hypertension and related diseases.

### CROSS-REFERENCE TO RELATED PATENT APPLICATIONS

This application claims the benefit under 35 U.S.C. §119(e) to U.S. Provisional Application No. 60/690,716 filed on Jun. 14, 2005, which is incorporated herein in its entirety by reference.

### FIELD

The invention relates generally to compounds and compositions useful in the treatment hypertension and related diseases. More specifically, the invention relates to nanoparticulate azelnidipine compositions having an effective average particle size of less than about 2000 nm. The invention also relates to methods of formulating and manufacturing nanoparticulate azelnidipine compositions, and to methods of treatment using the compositions.

### BACKGROUND OF THE INVENTION

The following discussion of the background of the invention is merely provided to aid the reader in understanding the invention and is not admitted to describe or constitute prior art to the invention.

### A. Background Regarding Azelnidipine

Hypertension, or high blood pressure, is known as “the silent killer” for two reasons. First, there are no specific symptoms. Estimates suggest that about one in three Americans has high blood pressure but is unaware of their condition. Second high blood pressure can lead to serious medical conditions that result in death. Such medical conditions include heart arrhythmias, heart attack, stroke and organ failure.

Unfortunately, the precise cause of hypertension in more than 90 percent of cases is unknown. Factors often associated with this type of “primary” or “essential” hypertension have been shown to include race, heredity, sex, age, obesity, drug use, physical activity and diet.

Occasionally, (e.g., in the remaining 10 percent of cases), hypertension is caused by some other physical problem such as atherosclerosis or cancer. This is termed “secondary” hypertension. Blood pressure may be restored to non-hypertensive levels if the primary problem is treated.

There are numerous classes of medication used to treat high blood pressure including centrally acting drugs, diuretics, angtiotensin converting enzyme (“ACE”) inhibitors, beta-blockers and calcium channel blockers (“CCBs”). CCBs work, in general terms, by blocking calcium channels and inhibiting the entry of calcium into the blood vessels and the heart tissue. The lowered calcium levels in the blood vessels and heart cause the blood vessels to dilate and the heart to beat more slowly, thereby lowering blood pressure. Some commercially available calcium channel blockers include Verapamil®, Diltiazem®, Nifedipine, Nicardipine®, Bepridil®, and Mibefradil.

Azelnidipine, another calcium channel blocker, is useful for the treatment of hypertension and related diseases. Azelnidipine is a dihydropyridine calcium channel antagonist with selectivity for L-type calcium channels. By inhibiting calcium channels, azelnidipine inhibits the influx of extracellular calcium through the L-type channel, resulting in relaxation of vascular smooth muscle and reduction in vascular resistance. Thus, azelnidipine functions as an antihypertensive agent.

Azelnidipine has the chemical name (±)-3-(1-Diphenylmethylazetidin-3-yl)-5-isopropyl 2-amino-1,4-dihydro-6-methyl-4-(3-nitrophenyl)-3,5-pyridinedicarboxylate. The empirical formula of azelnidipine is C₃₃H₃₄N₄O₆ and its molecular weight is 582.65. The structural formula of azelnidipine is:

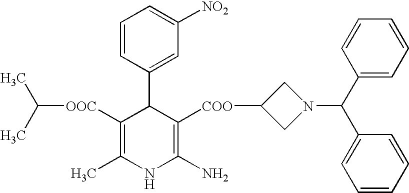

Azelnidipine is offered under the registered trademark CALBLOCK® by Sankyo Co. Ltd. of Japan. CALBLOCK® is offered as an oral tablet administered once daily for the treatment of hypertension and related diseases.

Azelnidipine is only slightly soluble in water; accordingly, absorption of azelnidipine may be increased when administered with a meal. Food delays gastric emptying thereby allowing more time for azelnidipine to dissolve. Additionally, food in the gastrointestinal system places the drug in contact with fat, a medium in which it is more soluble. Thus, conventional azelnidipine tablets should be taken with food, as the drug exhibits greater absorption and bioavailability when taken with food.

### B. Background Regarding Nanoparticulate Compositions

Nanoparticulate compositions, first described in U.S. Pat. No. 5,145,684 (“the '684 patent”), comprise particles of a poorly soluble therapeutic or diagnostic agent having adsorbed onto or associated with the surface thereof a non-crosslinked surface stabilizer. The '684 patent also describes method of making such nanoparticulate active agent compositions but does not describe compositions comprising azelnidipine in nanoparticulate form. Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;”5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” 6,432,381 for “Methods for Targeting Drug Delivery to the Upper and/or Lower Gastrointestinal Tract,” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” 6,656,504 for “Nanoparticulate Compositions Comprising Amorphous Cyclosporine;” 6,742,734 for “System and Method for Milling Materials;” 6,745,962 for “Small Scale Mill and Method Thereof;” 6,811,767 for “Liquid Droplet Aerosols of Nanoparticulate Drugs;” 6,908,626 for “Compositions Having a Combination of Immediate Release and Controlled Release Characteristics;” 6,969,529 for “Nanoparticulate Compositions Comprising Copolymers of Vinyl Pyrrolidone and Vinyl Acetate as Surface Stabilizers;” 6,976,647 for “System and Method for Milling Materials;” and 6,991,191 for “Method of Using a Small Scale Mill;” all of which are specifically incorporated by reference.

In addition, U.S. Patent Publication No. 20020012675 A1, for “Controlled Release Nanoparticulate Compositions;” U.S. Patent Publication No. 20050276974 for “Nanoparticulate Fibrate Formulations;” U.S. Patent Publication No. 20050238725 for “Nanoparticulate Compositions Having a Peptide as a Surface Stabilizer;” U.S. Patent Publication No. 20050233001 for “Nanoparticulate Megestrol Formulations;” U.S. Patent Publication No. 20050147664 for “Compositions Comprising Antibodies and Methods of Using the Same for Targeting Nanoparticulate Active Agent Delivery;” U.S. Patent Publication No. 20050063913 for “Novel Metaxalone Compositions;” U.S. Patent Publication No. 20050042177 for “Novel Compositions of Sildenafil Free Base;” U.S. Patent Publication No. 20050031691 for “Gel Stabilized Nanoparticulate Active Agent Compositions;” U.S. Patent Publication No. 20050019412 for “Novel Glipizide Compositions;” U.S. Patent Publication No. 20050004049 for “Novel Griseofulvin Compositions;” U.S. Patent Publication No. 20040258758 for “Nanoparticulate Topiramate Formulations;” U.S. Patent Publication No. 20040258757 for “Liquid Dosage Compositions of Stable Nanoparticulate Active Agents;” U.S. Patent Publication No. 20040229038 for “Nanoparticulate Meloxicam Formulations;” U.S. Patent Publication No. 20040208833 for “Novel Fluticasone Formulations;” U.S. Patent Publication No. 20040195413 for “Compositions and Method for Milling Materials;” U.S. Patent Publication No. 20040156895 for “Solid Dosage Forms Comprising Pullulan;” U.S. Patent Publication No. U.S. Patent Publication No. U.S. Patent Publication No. 20040156872 for “Novel Nimesulide Compositions;” U.S. Patent Publication No. 20040141925 for “Novel Triamcinolone Compositions;” U.S. Patent Publication No. 20040115134 for “Novel Nifedipine Compositions;” U.S. Patent Publication No. 20040105889 for “Low Viscosity Liquid Dosage Forms;” U.S. Patent Publication No. 20040105778 for “Gamma Irradiation of Solid Nanoparticulate Active Agents;” U.S. Patent Publication No. 20040101566 for “Novel benzoyl peroxide compositions;”U.S. Patent Publication No. 20040057905 for “Nanoparticulate Beclomethasone Dipropionate Compositions;” U.S. Patent Publication No. 20040033267 for “Nanoparticulate Compositions of Angiogenesis Inhibitors;” U.S. Patent Publication No. 20040033202 for “Nanoparticulate Sterol Formulations and Novel Sterol Combinations;” U.S. Patent Publication No. 20040018242 for “Nanoparticulate nystatin formulations;” U.S. Patent Publication No. 20040015134 for “Drug delivery Systems and Methods;” U.S. Patent Publication No. 20030232796 for “Nanoparticulate Polycosanol Formulations & Novel Polycosanol Combinations;” U.S. Patent Publication No. 20030215502 for “Fast Dissolving Dosage Forms Having Reduced Friability;” U.S. Patent Publication No. 20030185869 for “Nanoparticulate Compositions Having Lysozyme as a Surface Stabilizer;” U.S. Patent Publication No. 20030181411 for “Nanoparticulate Compositions of Mitogen-Activated Protein (MAP) Kinase Inhibitors;” U.S. Patent Publication No. 20030137067 for “Compositions Having a Combination of Immediate Release and Controlled Release Characteristics;” U.S. Patent Publication No. 20030108616 for “Nanoparticulate Compositions Comprising Copolymers of Vinyl Pyrrolidone and Vinyl Acetate as Surface Stabilizers;” U.S. Patent Publication No. 20030095928 for “Nanoparticulate Insulin;” U.S. Patent Publication No. 20030087308 for “Method for High Through-put Screening Using a Small Scale Mill or Microfluidics;” U.S. Patent Publication No. 20030023203 for “Drug Delivery Systems & Methods;” U.S. Patent Publication No. 20020179758 for “System and Method for Milling Materials;” and U.S. Patent Publication No. 20010053664 for “Apparatus for Sanitary Wet Milling,” describe nanoparticulate active agent compositions and are specifically incorporated by reference. None of these references describe compositions of nanoparticulate azelnidipine.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter,” all of which are specifically incorporated herein by reference.

Azelnidipine has high therapeutic value in the treatment of hypertension and related diseases. However, because it is practically insoluble in water, the dissolution of conventional microcrystalline azelnidipine tablets is reduced in the fasting state as compared to the fed state. Thus, azelnidipine has limited bioavailability in the fasted state as compared to the fed state, which limits the therapeutic outcome for all treatments requiring azelnidipine. Thus, there is a need in the art for azelnidipine formulations which overcome this and other problems associated with its use in the treatment of hypertension and related diseases.

There is a need for compositions of calcium channel blockers such as azelnidipine, that have enhanced bioavailability, increased dissolution rate, reduced drug dosage, and reduced adverse side effects. The present compositions and methods satisfy these needs.

### SUMMARY

The compositions and methods disclosed herein relate to compositions comprising at least one calcium channel blocker, such as azelnidipine or a salt or derivative thereof (referred to herein collectively as azelnidipine), having an effective average particle size of less than about 2000 nm. In one embodiment of the invention, the compositions also comprise at least one surface stabilizer. The compositions may be used to treat diseases or disorders such as, but not limited to hypertension, ischemic heart disease, stroke, peripheral artery disease, hypertensive heart disease, renal failure and combinations thereof.

Additionally, the compositions may comprise at least one primary and at least one secondary surface stabilizer. Exemplary surface stabilizers may include one or more of an anionic surface stabilizer, a cationic surface stabilizer, a non-ionic surface stabilizer, a zwitterionic surface stabilizers, and an ionic surface stabilizer.

In some embodiments, the compositions may additionally include one or more pharmaceutically acceptable excipients, carriers, active agents or combinations thereof. In some embodiments, active agents may includes agents useful for the treatment of hypertension, ischemic heart disease, stroke, peripheral artery disease, hypertensive heart disease, renal failure and combinations thereof. By way of example, but not by way of limitation, such active agents may include one or more of diuretics, beta blockers, ACE inhibitors, calcium channel blockers, alpha blockers, alpha-beta blockers, angiotensin antagonists, nervous system inhibitors, and vasodilators.

The nanoparticulate azelnidipine compositions described herein may be formulated for dosage or administration in a variety of forms. Although any pharmaceutically acceptable dosage form may be utilized, dosage forms contemplated include, but are not limited to formulations for oral, pulmonary, rectal, colonic, parenteral, intracisternal, intravaginal, intraperitoneal, ocular, otic, local, buccal, nasal, topical, liquid dispersions, gels, aerosols, ointments, creams, bioadhesives, lyophilized formulations, tablets, capsules, controlled release formulations, fast melt formulations, delayed release formulations, extended release formulations, pulsatile release formulations, mixed immediate release, controlled release formulations and combinations thereof. In some embodiments, solid dosages, such as an oral tablet, may be preferred.

The nanoparticulate azelnidipine compositions disclosed herein are also contemplated to exhibit improved pharmacokinetic properties as compared to a non-nanoparticulate composition of the same azelnidipine.

In further embodiments, the pharmacokinetic profiles of the nanoparticluate azelnidipine compositions may be substantially similar (e.g., are not significantly affected) when administered in the fed or fasted subject; in other embodiments, the nanoparticulate azelnidipine compositions may be bioequivalent when administered to a fed or fasted subject; in still other embodiments, the nanoparticulate azelnidipine compositions may not produce significantly different absorption levels when administered under fed versus fasted conditions.

Additionally disclosed are methods related to making nanoparticulate azelnidipine compositions having an effective average particle size of less than about 2000 nm. By way of example, but not by way of limitation, methods may include contacting particles of azelnidipine with at least one surface stabilizer for a time and under conditions sufficient to provide a nanoparticulate azelnidipine composition having an effective average particle size of less than about 2000 nm. In some methods, contacting may include grinding, wet grinding, homogenization, freezing, template emulsion, precipitation, supercritical fluid particle generation techniques and combinations thereof.

Also disclosed are methods of using the nanoparticulate azelnidipine formulations, for example, to treat or prevent diseases, disorders, symptoms or conditions in a subject. Exemplary methods may include administering to a subject a stable nanoparticulate azelnidipine composition including at least one azelnidipine or a salt or derivative thereof having an effective average particle size of less than about 2000 nm, and at least one surface stabilizer. In some embodiments, the subject may have been diagnosed with hypertension, ischemic heart disease, stroke, peripheral artery disease, hypertensive heart disease, renal failure or a combination thereof. In other methods, the compositions may be used to treat symptoms indicative of hypertension, ischemic heart disease, stroke, peripheral artery disease, hypertensive heart disease, renal failure or a combination thereof. Some treatment methods may include administering a composition including a nanoparticulate azelnidipine, at least one surface stabilizer and one or more active agents useful for the treatment hypertension and related disorders. By way of example, but not by way of limitation, such active agents may include one or more of diuretics, beta blockers, ACE inhibitors, calcium channel blockers, alpha blockers, alpha-beta blockers, angiotensin antagonists, nervous system inhibitors, and vasodilators. In some methods, the composition is administered in the form of an oral tablet.

Both the foregoing summary and the following brief description of the drawings and the detailed description are exemplary and explanatory and are intended to provide further details of the compositions and methods as claimed. Other objects, advantages, and novel features will be readily apparent to those skilled in the art from the following detailed description.

### BRIEF DESCRIPTION OF THE DRAWINGS

FIG. 1 shows a micrograph of a nanoparticulate azelnidipine formulation comprising azelnidipine, 5% w/w; hydroxypropyl cellulose (HPC-SL), 2% w/w; and deionised water, 93% w/w (Formulation 1, Table 1). Microscopy: 100×/1.40 oil phase objective. A 1 μm size reference is noted in the lower right corner.

FIG. 2 also shows a micrograph of nanoparticulate azelnidipine Formulation 1. Microscopy: 100×/1.40 Oil Phase objective. A 1 μm size reference is noted in the lower right corner.

FIG. 3 shows a micrograph of a nanoparticulate azelnidipine formulation comprising azelnidipine, 5% w/w; Plasdone S-630, 1.25% w/w; sodium lauryl sulfate, 0.05% w/w; deionised water 93.7%, w/w (Formulation 2, Table 1). Microscopy: 100×/1.40 oil phase objective. A 1 μm size reference is noted in the lower right corner.

FIG. 4 shows a micrograph of a nanoparticulate azelnidipine formulation comprising azelnidipine, 5% w/w; Lutrol F 108, 1.5% w/w; deionised water, 93.5% w/w (Formulation 6, Table 1). Microscopy: 100×/1.40 oil phase objective. A 1 μm size reference is noted in the lower right corner.

FIG. 5 shows a micrograph of a nanoparticulate azelnidipine formulation comprising azelnidipine, 5% w/w; Lutron F68, 1.25% w/w; docusate sodium, 0.5% w/w; deionised water, 93.7% w/w (Formulation 7, Table 1). Microscopy: 100× oil phase objective. A 1 μm size reference is noted in the lower right corner.

FIG. 6 also shows a micrograph of nanoparticulate azelnidipine Formulation 7. Microscopy: 100× oil phase objective. A 1 μm size reference is noted in the lower right corner.

FIG. 7 shows a micrograph of a nanoparticulate azelnidipine formulation comprising azelnidipine, 5% w/w; Plasdone K-17, 1.25% w/w; benzalkonium HCl, 0.05% w/w; deionised water 93.7% w/w (Formulation 8, Table 1). Microscopy: 100× oil phase objective. A 1 μm size reference is noted in the lower right corner.

FIG. 8 shows a micrograph of nanoparticulate azelnidipine Formulation 8; the micrograph was taken in the sample edge area. Microscopy: 100× oil phase objective. A 1 μm size reference is noted in the lower right corner.

### DETAILED DESCRIPTION

### A. Nanoparticulate Azelnidipine Compositions

The compositions of the invention comprise a calcium channel blocker such as azelnidipine or a salt or derivative thereof. The compositions comprise an azelnidipine, and preferably at least one surface stabilizer associated with or adsorbed on the surface of the drug. The azelnidipine particles may have an effective average particle size of less than about 2000 nm.

As taught by the '684 patent, and as exemplified in the examples below, not every combination of surface stabilizer and active agent will results in a stable nanoparticulate composition. It was surprisingly discovered that stable, nanoparticulate azelnidipine formulations can be made.

Advantages of the nanoparticulate azelnidipine formulation of the invention as compared to non-nanoparticulate azelnidipine compositions (e.g., microcrystalline or solubilized dosage forms) include, but are not limited to: (1) smaller tablet or other solid dosage form size; (2) smaller doses of drug required to obtain the same pharmacological effect; (3) improved pharmacokinetic profiles, (4) increased bioavailability; (5) substantially similar pharmacokinetic profiles of the azelnidipine compositions when administered in the fed versus the fasted state; (6) bioequivalency of the azelnidipine compositions when administered in the fed versus the fasted state; (7) an increased rate of dissolution for the azelnidipine compositions; and (8) the azelnidipine compositions can be used in conjunction with other active agents useful in the treatment of hypertension and related diseases, disorders, symptoms or conditions.

The present invention also relates to nanoparticulate azelnidipine compositions together with one or more non-toxic physiologically acceptable carriers, adjuvants, or vehicles, collectively referred to as carriers. The compositions may be formulated for parental injection (e.g., intravenous, intramuscular, or subcutaneous), oral administration in solid, liquid, bioadhesive or aerosol form, vaginal, nasal, rectal, ocular, local (powders, ointments, or drops), buccal, intracisternal, intraperitoneal, or topical administrations, and the like.

In some embodiments, a preferred dosage form may be a solid dosage form such as a tablet, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The present invention is described herein using several definitions, as set forth below and throughout the application.

The term “effective average particle size of less than about 2000 nm,” as used herein, means that at least about 50% of the nanoparticulate azelnidipine particles have a size of less than about 2000 nm (by weight or by other suitable measurement technique, such as by number or by volume) when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable nanoparticulate azelnidipine, “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase; (3) that the particles are chemically stable; and/or (4) where the azelnidipine has not been subject to a heating step at or above the melting point of the azelnidipine in the preparation of the nanoparticles of the present invention.

The term “conventional” or “non-nanoparticulate” active agent shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixtures thereof irrespective of their size, shape or morphology.

### B. Preferred Characteristics of the Nanoparticulate Azelnidipine Compositions

1. Increased Bioavailability

The compositions of the invention comprising a nanoparticulate azelnidipine, or a salt or derivative thereof, are proposed to exhibit increased bioavailability, and require smaller doses as compared to prior or conventional azelnidipine formulations.

In some embodiments, the nanoparticulate azelnidipine compositions, upon administration to a mammal, produce therapeutic results at a dosage which is less than that of a non-nanoparticulate dosage form of the same azelnidipine.

2. Improved Pharmacokinetic Profiles

The azelnidipine compositions described herein may also exhibit a desirable pharmacokinetic profile when administered to mammalian subjects. The desirable pharmacokinetic profile of the azelnidipine compositions preferably includes, but is not limited to: (1) a Cₘₐₓ for azelnidipine or a derivative or salt thereof, when assayed in the plasma of a mammalian subject following administration, that is preferably greater than the Cₘₐₓ for a non-nanoparticulate formulation of the same azelnidipine, administered at the same dosage; and/or (2) an AUC for azelnidipine or a derivative or a salt thereof, when assayed in the plasma of a mammalian subject following administration, that is preferably greater than the AUC for a non-nanoparticulate formulation of the same azelnidipine, administered at the same dosage; and/or (3) a Tₘₐₓ for azelnidipine or a derivative or a salt thereof, when assayed in the plasma of a mammalian subject following administration, that is preferably less than the Tₘₐₓ for a non-nanoparticulate formulation of the same azelnidipine, administered at the same dosage. The desirable pharmacokinetic profile, as used herein, is the pharmacokinetic profile measured after the initial dose of azelnidipine or derivative or a salt thereof.

In one embodiment, a composition comprising at least one nanoparticulate azelnidipine or a derivative or salt thereof exhibits in comparative pharmacokinetic testing with a non-nanoparticulate formulation of the same azelnidipine (e.g., CALBLOCK®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate azelnidipine formulation.

In another embodiment, the composition comprising at least one nanoparticulate azelnidipine or a derivative or salt thereof, exhibits in comparative pharmacokinetic testing with a non-nanoparticulate formulation of the same azelnidipine (e.g., CALBLOCK®), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate azelnidipine formulation.

In yet another embodiment, the composition comprising at least one nanoparticulate azelnidipine or a derivative or salt thereof, exhibits in comparative pharmacokinetic testing with a non-nanoparticulate formulation of the same azelnidipine (e.g., CALBLOCK®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate azelnidipine formulation.

3. The Pharmacokinetic Profiles of the Azelnidipine Compositions are Not Affected by the Fed or Fasted State of the Subject Ingesting the Compositions

In one embodiment of the invention, the pharmacokinetic profile of the nanoparticulate azelnidipine compositions are not substantially affected by the fed or fasted state of a subject ingesting the composition. This means that there would be little or no appreciable difference in the quantity of drug absorbed or the rate of drug absorption when the nanoparticulate azelnidipine compositions are administered in the fed or fasted state.

For conventional azelnidipine formulations, i.e., CALBLOCK®, the absorption of azelnidipine is increased when administered with food. This difference in absorption observed with conventional azelnidipine formulations is undesirable. The nanoparticulate azelnidipine formulations described herein are proposed to overcome this problem, as the azelnidipine formulations are likely to reduce or preferably substantially eliminate significantly different absorption levels when administered under fed as compared to fasting conditions.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed.

4. Bioequivalency of Azelnidipine Compositions When Administered in the Fed Versus the Fasted State

In one embodiment of the invention, administration of a nanoparticulate azelnidipine composition to a subject in a fasted state is bioequivalent to administration of the composition to a subject in a fed state. The difference in absorption of the nanoparticulate azelnidipine compositions, when administered in the fed versus the fasted state, preferably is less than about 100%, less than about 90%, less than about 80%, less than about 70%, less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In some embodiments, the invention encompasses compositions comprising at least one nanoparticulate azelnidipine, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Azelnidipine Compositions

The nanoparticulate azelnidipine compositions are proposed to have unexpectedly dramatic dissolution profiles. Rapid dissolution of an administered active agent is preferable, as faster dissolution generally leads to faster onset of action and greater bioavailability. To improve the dissolution profile and bioavailability of the azelnidipine, it would be useful to increase the drug's dissolution so that it could attain a level close to 100%.

The azelnidipine compositions of the invention preferably have a dissolution profile in which within about 5 minutes at least about 20% of the composition is dissolved. In other embodiments, at least about 30% or at least about 40% of the azelnidipine composition is dissolved within about 5 minutes. In yet other embodiments, preferably at least about 40%, at least about 50%, at least about 60%, at least about 70%, or at least about 80% of the azelnidipine composition is dissolved within about 10 minutes. In further embodiments, preferably at least about 70%, at least about 80%, at least about 90%, or at least about 100% of the azelnidipine composition is dissolved within 20 minutes.

In some embodiments, dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersibility of the Azelnidipine Compositions of the Invention

An additional feature of the azelnidipine compositions described herein may include redispersion such that the effective average particle size of the redispersed azelnidipine particles is less than about 2 microns. This is significant, as if upon administration the azelnidipine compositions of the invention did not redisperse to a substantially nanoparticulate size, then the dosage form may lose the benefits afforded by formulating the azelnidipine into a nanoparticulate size.

Not wishing to be bound by any theory, it is proposed that nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall.

Moreover, the nanoparticulate azelnidipine compositions of the invention exhibit dramatic redispersion of the nanoparticulate azelnidipine particles upon administration to a mammal, such as a human or animal, as demonstrated by reconstitution/redispersion in a biorelevant aqueous media such that the effective average particle size of the redispersed azelnidipine particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, water, aqueous electrolyte solutions or aqueous solutions of any salt, acid, or base, or a combination thereof, which exhibit the desired pH and ionic strength. Such redispersion in a biorelevant media is predictive of in vivo efficacy of the azelnidipine dosage form.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed azelnidipine particles of the invention (redispersed in water, a biorelevant medium, or any other suitable dispersion medium) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

In still other embodiments, the redispersed azelnidipine particles, when administered to a mammal, redisperse such that the particles have an effective average particle size of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Azelnidipine Compositions Used in Conjunction with Other Active Agents

The compositions comprising a nanoparticuulate azelnidipine, or a salt or derivative thereof, can additionally comprise one or more compounds useful in the treatment of hypertension and related diseases, or the azelnidipine compositions can be administered in conjunction with such a compound. Examples of such compounds include, but are not limited to diuretics, beta blockers, ACE inhibitors, calcium channel blockers, alpha blockers, alpha-beta blockers, angiotensin antagonists, nervous system inhibitors, vasodilators, antihypertensive agents, and blood lipid-lowering agents.

### C. Nanoparticulate Azelnidipine Compositions

The invention provides compositions comprising azelnidipine particles and at least one surface stabilizer. The surface stabilizers preferably are adsorbed on, or associated with, the surface of the azelnidipine particles. In some embodiments, surface stabilizers preferably physically adhere on, or associate with, the surface of the nanoparticulate azelnidipine particles, but do not chemically react with the azelnidipine particles or itself. Individually adsorbed molecules of the surface stabilizer are essentially free of intermolecular cross-linkages.

The present invention also includes azelnidipine compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

1. Azelnidipine Particles

The compositions of the invention comprise particles of azelnidipine or a salt or derivative thereof. The particles can be in crystalline phase, semi-crystalline phase, amorphous phase, semi-amorphous phase, or a combination thereof.

2. Surface Stabilizers

The choice of a surface stabilizer for an azelnidipine is non-trivial and required extensive experimentation to realize a desirable formulation. Accordingly, the present invention is directed to the surprising discovery that nanoparticulate azelnidipine compositions can be made.

Combinations of more than one surface stabilizers can be used in the invention. Suitable surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate (dioctyl sodium sulfosuccinate), gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween® 20 and Tween® 80 (ICI Specialty Chemicals)); polyethylene glycols (e.g., Carbowaxs® 3550 and 934 (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,3,3-tetramethylbutyl)-phenol polymer with ethylene oxide and formaldehyde (also known as tyloxapol, superione, and triton), poloxamers (e.g., Pluronics® F68 and F108, which are block copolymers of ethylene oxide and propylene oxide); poloxamines (e.g., Tetronic® 908, also known as Poloxamine™ 908, which is a tetrafunctional block copolymer derived from sequential addition of propylene oxide and ethylene oxide to ethylenediamine (BASF Wyandotte Corporation, Parsippany, N.J.)); Tetronic® 1508 (T-1508) (BASF Wyandotte Corporation), Tritons® X-200, which is an alkyl aryl polyether sulfonate (Rohm and Haas); Crodestas™ F-110, which is a mixture of sucrose stearate and sucrose distearate (Croda Inc.); p-isononylphenoxypoly-(glycidol), also known as Olin®-1OG or Surfactant™ 10-G (Olin Chemicals, Stamford, Conn.); Crodestas™ SL-40 (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUATT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants Physical Chemistry (Marcel Dekker, 1991); and J. Richmond, Cationic Surfactants Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

In some embodiments, the surface stabilizers are copovidone (e.g., Plasdone S630, which is random copolymer of vinyl acetate and vinyl pyrrolidone) and docusate sodium.

The surface stabilizers are commercially available and/or can be prepared by techniques known in the art. See e.g., Handbook of Pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, include colloidal silicon dioxide, such as Aerosil® 200, talc, stearic acid, magnesium stearate, calcium stearate, and silica gel.

Examples of sweeteners include any natural or artificial sweetener, such as sucrose, xylitol, sodium saccharin, cyclamate, aspartame, and acsulfame. Examples of flavoring agents include Magnasweet® (trademark of MAFCO), bubble gum flavor, and fruit flavors, and the like.

Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of buffers include phosphate buffer, citrate buffers and buffers made from other organic acids.

Examples of wetting or dispersing agents include a naturally-occurring phosphatide, for example, lecithin or condensation products of n-alkylene oxide with fatty acids, for example, polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyethylene sorbitan monooleate.

Examples of effervescent agents include effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Azelnidipine Particle Size

The compositions of the invention comprise nanoparticulate azelnidipine particles which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the azelnidipine particles have a particle size of less than the effective average, by weight (or by other suitable measurement technique, such as by volume, number, etc.), i.e., less than about 2000 nm, 1900 nm, 1800 nm, etc., when measured by the above-noted techniques. Preferably, at least about 70%, about 90%, or about 95% of the azelnidipine particles have a particle size of less than the effective average, i.e., less than about 2000 nm, 1900 nm, 1800 nm, 1700 nm, etc.

In the present invention, the value for D50 of a nanoparticulate azelnidipine composition is the particle size below which 50% of the azelnidipine particles fall, by weight (or by other suitable measurement technique, such as by volume, number, etc.). Similarly, D90 is the particle size below which 90% of the azelnidipine particles fall, by weight (or by other suitable measurement technique, such as by volume, number, etc.).

5. Concentration of Azelnidipine and Surface Stabilizers

The relative amounts of azelnidipine, or a salt or derivative thereof, and one or more surface stabilizers may vary. The optimal amount of the individual components can depend, for example, upon the particular azelnidipine selected, the hydrophilic lipophilic balance (HLB), melting point, and the surface tension of water solutions of the stabilizer, etc.

In some embodiments, the concentration of the azelnidipine may vary from about 99.5% to about 0.001%, from about 95% to about 0.1%, or from about 90% to about 0.5%, by weight, based on the total combined dry weight of the azelnidipine and at least one surface stabilizer, not including other excipients.

In other embodiments, the concentration of the at least one surface stabilizer may vary from about 0.5% to about 99.999%, from about 5.0% to about 99.9%, or from about 10% to about 99.5%, by weight, based on the total combined dry weight of the azelnidipine and at least one surface stabilizer, not including other excipients.

6. Exemplary Nanoparticulate Azelnidipine Tablet Formulations

Several exemplary azelnidipine tablet formulations are given below. These examples are not intended to limit the claims in any respect, but rather to provide exemplary tablet formulations of azelnidipine which can be utilized in the methods of the invention. Such exemplary tablets can also comprise a coating agent.

Exemplary Nanoparticulate
Azelnidipine Tablet Formulation #1

| Component | g/Kg |
|---|---|
| Azelnidipine | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate
Azelnidipine Tablet Formulation #2

| Component | g/Kg |
|---|---|
| Azelnidipine | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate
Azelnidipine Tablet Formulation #3

| Component | g/Kg |
|---|---|
| Azelnidipine | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate
Azelnidipine Tablet Formulation #4

| Component | g/Kg |
|---|---|
| Azelnidipine | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

### D. Methods of Making Nanoparticulate Azelnidipine Compositions

The nanoparticulate azelnidipine compositions can be made using, for example, milling, homogenization, precipitation, freezing, supercritical particle generation, or template emulsion techniques. Exemplary methods of making nanoparticulate compositions are described in the '684 patent. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate azelnidipine compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Azelnidipine Dispersions

Milling an azelnidipine, or a salt or derivative thereof, to obtain a nanoparticulate dispersion comprises dispersing the azelnidipine particles in a liquid dispersion medium in which the azelnidipine is poorly soluble, followed by applying mechanical means in the presence of grinding media to reduce the particle size of the azelnidipine to the desired effective average particle size. The dispersion medium can be, for example, water, safflower oil, ethanol, t-butanol, glycerin, polyethylene glycol (PEG), hexane, or glycol. In some embodiments, a preferred dispersion medium is water.

The azelnidipine particles can be reduced in size in the presence of at least one surface stabilizer. Alternatively, azelnidipine particles can be contacted with one or more surface stabilizers after attrition. Other compounds, such as a diluent, can be added to the azelnidipine/surface stabilizer composition during the size reduction process. Dispersions can be manufactured continuously or in a batch mode.

2. Precipitation to Obtain Nanoparticulate Azelnidipine Compositions

Another method of forming the desired nanoparticulate azelnidipine compositions is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the azelnidipine in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Azelnidipine Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of an azelnidipine, or a salt or derivative thereof, in a liquid dispersion medium, followed by subjecting the dispersion to homogenization to reduce the particle size of an azelnidipine to the desired effective average particle size. The azelnidipine particles can be reduced in size in the presence of at least one surface stabilizer. Alternatively, the azelnidipine particles can be contacted with one or more surface stabilizers either before or after attrition. Other compounds, such as a diluent, can be added to the azelnidipine/surface stabilizer composition either before, during, or after the size reduction process. Dispersions can be manufactured continuously or in a batch mode.

4. Cryogenic Methodologies to Obtain Nanoparticulate Azelnidipine Compositions

Another method of forming the desired nanoparticulate azelnidipine compositions is by spray freezing into liquid (SFL). This technology comprises an organic or organoaqueous solution of azelnidipine with stabilizers, which is injected into a cryogenic liquid, such as liquid nitrogen. The droplets of the azelnidipine solution freeze at a rate sufficient to minimize crystallization and particle growth, thus formulating nanostructured azelnidipine particles. Depending on the choice of solvent system and processing conditions, the nanoparticulate azelnidipine particles can have varying particle morphology. In the isolation step, the nitrogen and solvent are removed under conditions that avoid agglomeration or ripening of the azelnidipine particles.

As a complementary technology to SFL, ultra rapid freezing (URF) may also be used to created equivalent nanostructured azelnidipine particles with greatly enhanced surface area.

URF comprises an organic or organoaqueous solution of azelnidipine with stabilizers onto a cryogenic substrate.

5. Emulsion Methodologies to Obtain Nanoparticulate Azelnidipine Compositions

Another method of forming the desired nanoparticulate azelnidipine, or a salt or derivative thereof, composition is by template emulsion. Template emulsion creates nanostructured azelnidipine particles with controlled particle size distribution and rapid dissolution performance. The method comprises an oil-in-water emulsion that is prepared, then swelled with a non-aqueous solution comprising the azelnidipine and stabilizers. The particle size distribution of the azelnidipine particles is a direct result of the size of the emulsion droplets prior to loading with the azelnidipine a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured azelnidipine particles are recovered. Various azelnidipine particles morphologies can be achieved by appropriate control of processing conditions.

6. Supercritical Fluid Techniques Used to Obtain Nanoparticulate Azelnidipine Compositions

Published International Patent Application No. WO 97/144407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers.

### E. Methods of Using the Nanoparticulate Azelnidipine Compositions of the Invention

The invention provides a method of rapidly increasing the bioavailability (e.g., plasma levels) of azelnidipine in a subject. Such a method comprises orally administering to a subject an effective amount of a composition comprising an azelnidipine. In some embodiments, the azelnidipine compositions, in accordance with standard pharmacokinetic practice, have a bioavailability that is about 50% greater, about 40% greater, about 30% greater, about 20% greater or about 10% greater than a conventional dosage form. Additionally, when tested in fasting subjects in accordance with standard pharmacokinetic practice, the nanoparticulate azelnidipine compositions produce a maximum blood plasma concentration profile in less than about 6 hours, less than about 5 hours, less than about 4 hours, less than about 3 hours, less than about 2 hours, less than about 1 hour, or less than about 30 minutes after the initial dose of the compositions.

The compositions of the invention may be useful in the treatment of hypertension and related diseases. Diseases related to hypertension include, but are not limited to, ischemic heart disease, stroke, peripheral artery disease, hypertensive heart disease, and renal failure.

The azelnidipine compounds of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), as a bioadhesive, or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate azelnidipine, or a salt or derivative thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to an azelnidipine, the liquid dosage forms may comprise inert diluents commonly used in the art, such as water or other solvents, solubilizing agents, and emulsifiers.

Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

‘Therapeutically effective amount’ as used herein with respect to an azelnidipine, dosage shall mean that dosage that provides the specific pharmacological response for which an azelnidipine is administered in a significant number of subjects in need of such treatment. It is emphasized that ‘therapeutically effective amount,’ administered to a particular subject in a particular instance will not always be effective in treating the diseases described herein, even though such dosage is deemed a ‘therapeutically effective amount’ by those skilled in the art. It is to be further understood that azelnidipine dosages are, in particular instances, measured as oral dosages, or with reference to drug levels as measured in blood.

One of ordinary skill will appreciate that effective amounts of an azelnidipine can be determined empirically and can be employed in pure form or, where such forms exist, in pharmaceutically acceptable salt, ester, or prodrug form. Actual dosage levels of an azelnidipine in the nanoparticulate compositions of the invention may be varied to obtain an amount of an azelnidipine that is effective to obtain a desired therapeutic response for a particular composition and method of administration. The selected dosage level therefore depends upon the desired therapeutic effect, the route of administration, the potency of the administered azelnidipine, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### F. Examples

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1

The purpose of this example was to demonstrate the preparation of compositions comprising nanoparticulate azelnidipine or a salt or derivative thereof.

Eight different azelnidipine formulations, detailed below in Table 1, Column 2, were synthesized and evaluated as follows. The formulations comprising azelnidipine were milled in the 10 ml chamber of a NanoMill® 0.01 (NanoMill Systems, King of Prussia, Pa.; see e.g., U.S. Pat. No. 6,431,478) along with 500 micron PolyMill® attrition media (Dow Chemical Co.), at a media load of about 89%. Formulations 2-8 were milled at a speed of 3000 RPM for 60 minutes; formulation 1 was milled at 2500 RPM for 60 minutes.

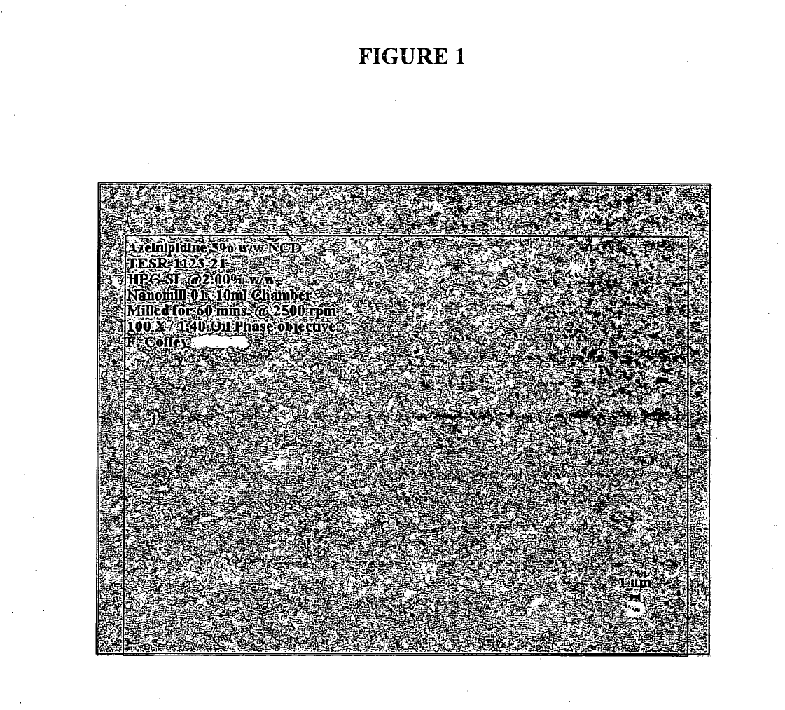

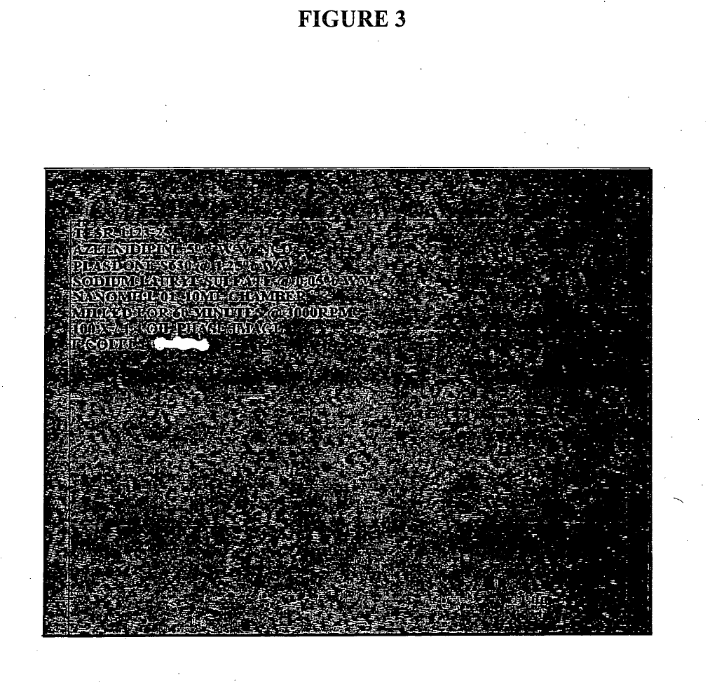

Following milling, the azelnidipine particles were evaluated using a Lecia DM5000B microscope and Lecia CTR 5000 light source (Laboratory Instruments & Supplies (I) Ltd. Ashbourne CO MEATH ROI). Observations are presented in Table 1, Column 3. Successful formulations, as determined by microscopy observation, are noted in column 4 (“Y” indicates “YES” the formulation was successful; “N” indicates “NO” the formulation was not successful). Micrographs of Formulations 1, 2, 6, 7 and 8 are shown in FIGS. 1-8. Additionally or alternatively, the particle size of the milled azelnidipine particles may be measured, using deionized, distilled water and a Horiba LA 910 particle size analyzer. After particle size analysis, a “successful composition,” may define formulations in which the initial mean and/or D50 milled azelnidipine particle size is less than about 2000 nm. Particles may additionally be analyzed before and after a 60 second sonication.

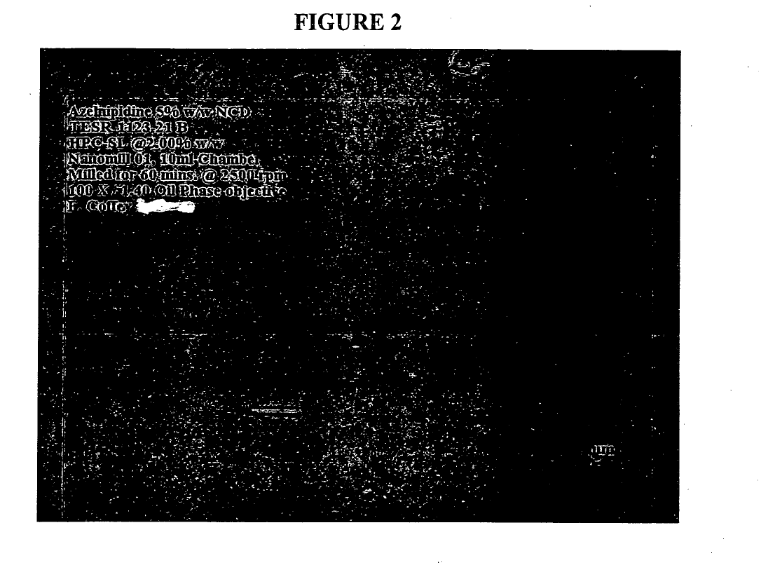

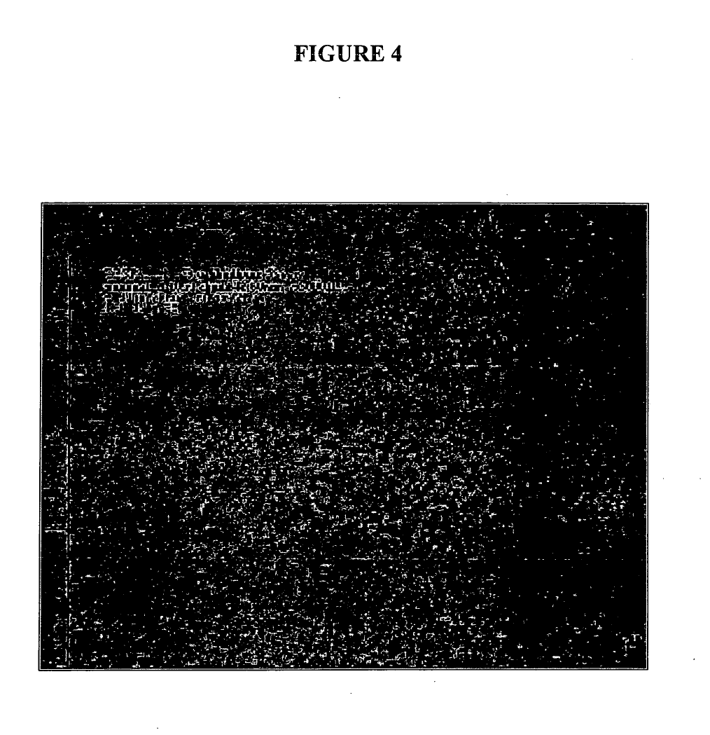

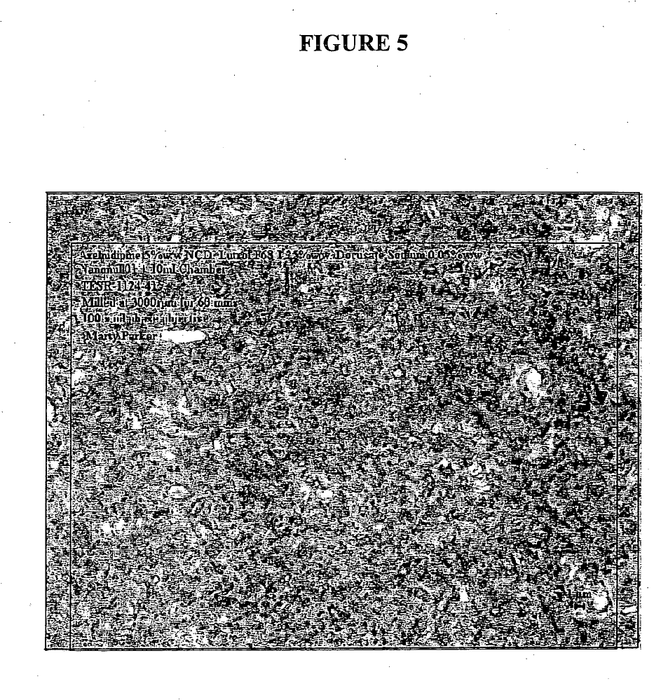

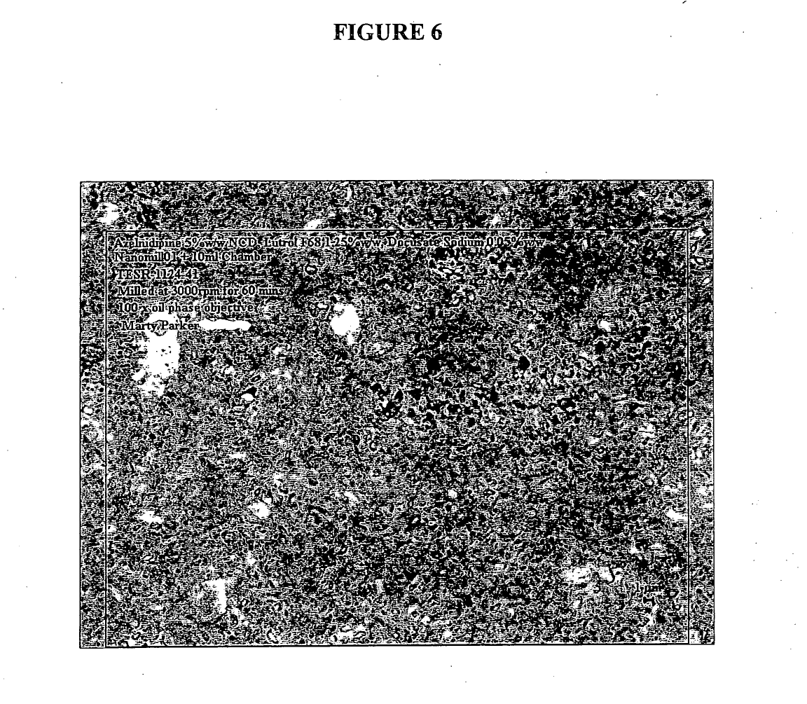

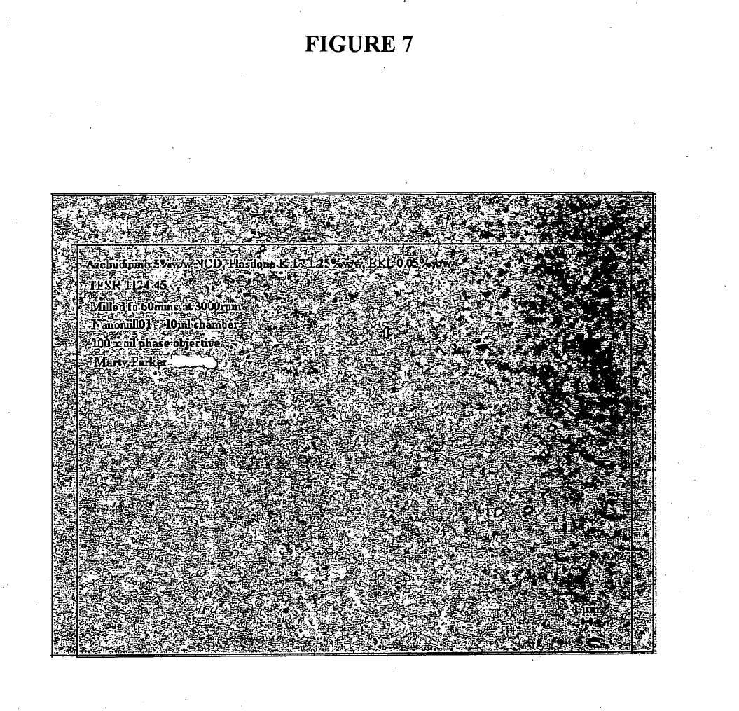

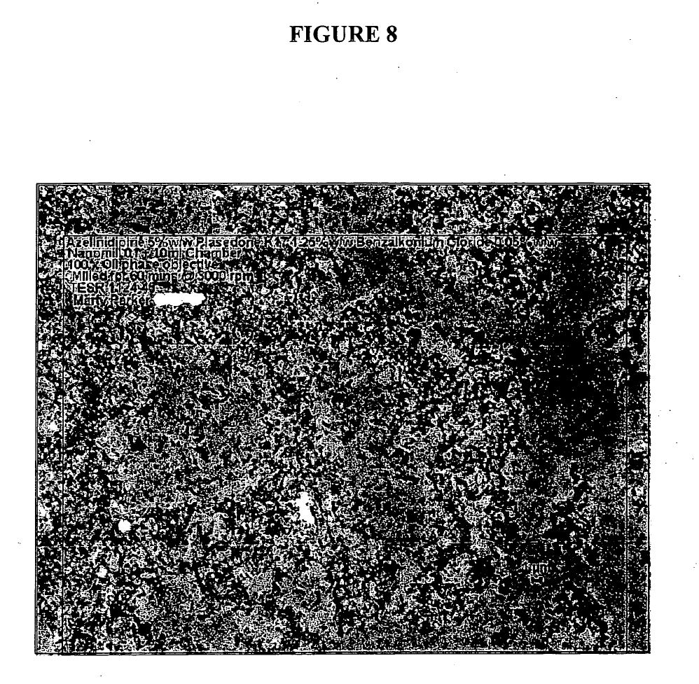

TABLE 1

| | | | Successful |
|---|---|---|---|
| Sample | Formulation | Microscopy Observation | formulation |
| 1 | Azelnidipine, | Nanoparticles of azelnidipine were | Y |
| | 5% w/w | observed which displayed clear | |
| | HPC-SL, 2% w/w | evidence of Brownian motion. Some | |
| | Deionised Water, | isolated rod-like crystals were also | |
| | 93% w/w | evident which may be partially milled | |
| | | material. The azelnidipine | |
| | | nanoparticles observed appeared to be | |
| | | very small (well below 1 micron). The | |
| | | majority of the azelnidipine | |
| | | nanoparticles appeared to be below | |
| | | 2000 nm. See FIGS. 1 and 2. | |
| 2 | Azelnidipine, | Nanoparticles of azelnidipine were | Y |
| | 5% w/w | observed and Brownian motion was | |
| | Plasdone S-630, | also evident. Some larger particles | |
| | 1.25% w/w | were also observed which may be | |
| | Sodium Lauryl | unmilled/partially milled material or | |
| | Sulfate, 0.05% w/w | agglomeration. The majority of the | |
| | Deionised Water, | azelnidipine particles present appeared | |
| | 93.7% w/w | to be well below the acceptance | |
| | | criteria of below 2000 nm. See FIG. | |
| | | 3. | |
| 3 | Azelnidipine, | Some azelnidipine nanoparticles were | N |
| | 5% w/w | present and a little Brownian motion | |
| | Pharmacoat 603, | was observed. However, the majority | |
| | 1.25% w/w | of the slide showed evidence of severe | |
| | Docusate sodium, | flocculation or agglomeration. The | |
| | 0.05% w/w | microphotograph is representative of | |
| | Deionised Water, | the entire sample as the | |
| | 93.7% w/w | flocculation/agglomeration was | |
| | | present throughout the sample. | |
| 4 | Azelnidipine, | Results show severe flocculation with | N |
| | 5% w/w | no Brownian motion visible. | |
| | Tyloxapol, | | |
| | 1.25% w/w | | |
| | Deionised Water, | | |
| | 93.75% w/w | | |
| 5 | Azelnidipine, | The results of the microscopy | N |
| | 5% w/w | observation show severe | |
| | Tween 80, | flocculation/aggregation with very | |
| | 1.25% w/w | little Brownian motion present. | |
| | Deionised Water, | | |
| | 93.75% w/w | | |
| 6 | Azelnidipine, | The sample exhibited Brownian | Y |
| | 5% w/w | motion evidently, and looked | |
| | Lutrol F108, | homogeneous throughout the slide. | |
| | 1.5% w/w | Individually dispersed azelnidipine | |
| | Deionised Water, | nanoparticles were clearly evident. | |
| | 93.5% w/w | Some larger particles with a needle | |
| | | like shape were seen (these did not | |
| | | seem to constitute the majority of the | |
| | | sample) and may be partially milled or | |
| | | may be unmilled azelnidipine | |
| | | particles. See FIG. 4. | |
| 7 | Azelnidipine, | Microscopy showed the sample to be | Y |
| | 5% w/w | well dispersed with azelnidipine | |
| | Lutrol F68, | nanoparticles clearly visible. | |
| | 1.25% w/w | Brownian motion was also observed. | |
| | Docusate sodium, | There was no sign of flocculation. | |
| | 0.05% w/w | Azelnidipine crystals were observed | |
| | Deionised Water, | throughout the sample these seemed to | |
| | 93.7% w/w | be needle like in shape which may | |
| | | indicate the presence of some partially | |
| | | milled drug. See FIGS. 5 and 6. | |
| 8 | Azelnidipine, | Microscopy showed the sample to be | Y |
| | 5% w/w | well dispersed with azelnidipine | |
| | Plasdone K-17, | nanoparticles visible exhibiting | |
| | 1.25% w/w | Brownian motion. There was some | |
| | Benzalkonium | signs of flocculation particularly | |
| | HCl, 0.05% w/w | towards the top of the slide sample. | |
| | Deionised Water, | Some azelnidipine crystals were also | |
| | 93.7% w/w | visible, this may indicate the presence | |
| | | of some partially milled azelnidipine | |
| | | particles. Such flocculation appeared | |
| | | to take place mainly at the borderline | |
| | | of the slide cover and could be due to | |
| | | sample drying out at the edges of the | |
| | | slide. Indeed, very little flocculation | |
| | | was seen in the central area of the | |
| | | slide. See FIGS. 7 and 8. | |

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

All references, patents, and/or applications cited in the specification are incorporated by reference in their entireties, including any tables and figures, to the same extent as if each reference had been incorporated by reference in its entirety individually.

1. A stable nanoparticulate azelnidipine composition comprising:
(a) particles of an azelnidipine or a salt having an average effective particle size of less than about 2000 nm; and
(b) at least one surface stabilizer.

2. The composition of claim 1, wherein azelnidipine is in a crystalline phase, an amorphous phase, or a semi-crystalline phase.

3. The composition of claim 1, wherein the effective average particle size of the azelnidipine particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1, wherein the composition is formulated:
(a) for administration selected from the group consisting of oral, pulmonary, intravenous, rectal, ophthalmic, colonic, parenteral, intracisternal, intravaginal, intraperitoneal, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, mixed immediate release formulations, controlled release formulations; or
(d) any combination of (a), (b), and (c).

5. The composition of claim 1, wherein the composition further comprises one or more pharmaceutically acceptable excipients, carriers, or a combination thereof.

6. The composition of claim 1, additionally comprising one or more active agents useful for the treatment of hypertension and related diseases.

7. The composition of claim 6, wherein said related disease is selected from the group consisting of ischemic heart disease, stroke, peripheral artery disease, hypertensive heart disease, renal failure and combinations thereof.

8. The composition of claim 6, wherein said one or more active agents is selected from the group consisting of diuretics, beta blockers, ACE inhibitors, calcium channel blockers, alpha blockers, alpha-beta blockers, angiotensin antagonists, nervous system inhibitors, and vasodilators.

9. The composition of claim 1, wherein
(a) the amount of azelnidipine is selected from the group consisting of from about 99.5% to about 0.001%, from about 95% to about 0.1%, and from about 90% to about 0.5%, by weight, based on the total combined weight of azelnidipine and at least one surface stabilizer, not including other excipients;
(b) at least one surface stabilizer is present in an amount selected from the group consisting of from about 0.5% to about 99.999% by weight, from about 5.0% to about 99.9% by weight, and from about 10% to about 99.5% by weight, based on the total combined dry weight of azelnidipine and at least one surface stabilizer, not including other excipients; or
(c) a combination of (a) and (b).

10. The composition of claim 1, further comprising at least one primary surface stabilizer and at least one secondary surface stabilizer.

11. The composition of claim 1, wherein the surface stabilizer is selected from the group consisting of an anionic surface stabilizer, a cationic surface stabilizer, a zwitterionic surface stabilizer, a non-ionic surface stabilizer, and an ionic surface stabilizer.

12. The composition of claim 1, wherein at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate (dioctyl sodium sulfosuccinate), dialkylesters of sodium sulfosuccinic acid, sodium lauryl sulfate, alkyl aryl polyether sulfonates, mixtures of sucrose stearate and sucrose distearate, C₁₈H₃₇CH₂C(O)N(CH₃)—CH₂(CHOH)₄(CH₂OH)₂, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethyl ammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquaternium 10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, quaternized ammonium salt polymers, alkyl pyridinium salts; amines, amine salts, amine oxides, imide azolinium salts, protonated quaternary acrylamides, methylated quaternary polymers, and cationic guar.

13. The composition of claim 1, wherein the pharmacokinetic profile of said composition is not significantly affected by the fed or fasted state of a subject ingesting said composition.

14. The composition of claim 1 which does not produce significantly different absorption levels when administered under fed as compared to fasting conditions.

15. The composition of claim 14, wherein the difference in absorption of the active agent composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

16. The composition of claim 1, wherein administration of the composition to a subject in a fasted state is bioequivalent to administration of said composition to a subject in a fed state.

17. The composition of claim 16, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

18. The composition of claim 1, wherein:
(a) the Tₘₐₓ of the azelnidipine, when assayed in the plasma of a mammalian subject following administration, is less than the Tₘₐₓ for a non-nanoparticulate composition of the same azelnidipine, administered at the same dosage;
(b) the Cₘₐₓ of the azelnidipine, when assayed in the plasma of a mammalian subject following administration, is greater than the Cₘₐₓ for a non-nanoparticulate composition of the same azelnidipine, administered at the same dosage;
(c) the AUC of the azelnidipine, when assayed in the plasma of a mammalian subject following administration, is greater than the AUC for a non-nanoparticulate composition of the same azelnidipine, administered at the same dosage; or
(d) any combination of (a), (b), and (c).

19. The composition of claim 18, wherein:
(a) the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate composition of the same azelnidipine, administered at the same dosage;
(b) the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate composition of the same azelnidipine, administered at the same dosage;
(c) the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of the same azelnidipine, administered at the same dosage; or
(d) any combination of (a), (b), and (c).

20. The composition claim 1, wherein:
(a) upon administration to a mammal the azelnidipine particles redisperse such that the particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm;
(b) the composition redisperses in a biorelevant media such that the azelnidipine particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm; or
(c) a combination of (a) and (b).

21. The composition of claim 20, wherein the biorelevant media is selected from the group consisting of water, aqueous electrolyte solutions, aqueous solutions of a salt, aqueous solutions of an acid, aqueous solutions of a base, and combinations thereof.

22. A method of preparing a nanoparticulate azelnidipine, or a salt or derivative thereof, comprising contacting particles of an azelnidipine with at least one surface stabilizer for a time and under conditions sufficient to provide a nanoparticulate azelnidipine composition having an effective average particle size of less than about 2000 nm.

23. The method of claim 22, wherein the contacting comprises grinding, wet grinding, homogenization, freezing, emulsion techniques, supercritical fluid particle generation techniques, precipitation, or a combination thereof.

24. A method for the treatment of hypertension or a related disease in a subject comprising administering to a subject of an effective amount of a composition comprising:
(a) particles of azelnidipine or salt or derivative thereof having an average effective particle size of less than about 2000 nm; and
(b) at least one surface stabilizer.

25. The method of claim 24, further comprising one or more active agents useful for the treatment of hypertension and related diseases.

26. The method of claim 25, wherein the related disease is selected from the group consisting of ischemic heart disease, stroke, peripheral artery disease, hypertensive heart disease, renal failure and a combination thereof.

27. The method of claim 25, wherein the one or more active agents is selected from the group consisting of diuretics, beta blockers, ACE inhibitors, calcium channel blockers, alpha blockers, alpha-beta blockers, angiotensin antagonists, nervous system inhibitors, and vasodilators.

28. The method of claim 24, wherein the composition is in the form of an oral tablet.

29. The composition of claim 1, wherein at least one surface stabilizer is selected from the group consisting of hydroxypropylcellulose, a copolymer of vinylpyrrolidone and vinyl acetate, sodium lauryl sulfate, hypromellose, poloxamer, docusate sodium, polyvinylpyrrolidone, and benzalkonium chloride.
